(19) United States
(12) Patent Application Publication
(10) Pub. No.: US 2024/0151701 A1
(43) Pub. Date: May 9, 2024

(54) ANALYSIS OF MILK

(71) Applicant: GEA Farm Technologies GmbH, Bönen (DE)

(72) Inventors: Peter Peters, Hamm (DE); Christoph Zenz, Bochum (DE); Martin Wortmann, Recklinghausen (DE)

(21) Appl. No.: 18/281,090

(22) PCT Filed: Mar. 2, 2022
(86) PCT No.: PCT/EP2022/055273

§ 371 Date: Sep. 8, 2023

(30) Foreign Application Priority Data
Mar. 9, 2021 (DE) 10 2021 105 644.6

(51) Int. Cl. G01N 33/04; G01N 21/85

(52) U.S. Cl. CPC G01N 33/04; G01N 21/85

(57) ABSTRACT

The invention relates to a device (1) for analyzing milk, comprising: a line section (2) for the milk, a light source unit (4) which emits light into the line section (2), a detection unit (5) for detecting light exiting from the line section (2), and in particular an evaluation unit (6) which is designed to analyze the milk in relation to the ingredients based on signals of the detection unit (5).

The invention relates to the analysis of milk, in particular an apparatus, an arrangement and a method therefor.

Raw milk is an important food and an important raw material for the food industry. In order to protect the consumer, for the purpose of technical processability, and also for market control purposes, raw milk must meet certain national and international quality requirements.

In milking apparatuses and methods in general, and in particular in automatic and automated milking using semi- and also fully automatic milking systems, extended functions play an important part. In particular, special emphasis is placed on ensuring quality standards of the milk, in particular also checking for obviously altered milk.

It is known for milk obtained by milking to be analyzed spectroscopically with regard to constituents indirectly or directly after a milking operation. In this case, the characteristic absorption spectrum of the constituents is utilized. If light of a specific wavelength is guided into the milk, it is absorbed by the milk, provided that the milk comprises a constituent that absorbs this wavelength.

It is known from the prior art to use LEDs having a fixed wavelength for spectroscopic analysis. That is possible with simple components which, besides the single LED, merely require a light sensor and simple evaluation electronics. However, such a component is only sensitive to a specific constituent. Therefore, as early as when designing the component it is necessary to stipulate which constituent this ought to be. The analysis of a plurality of different constituents requires a relatively high outlay in accordance with the prior art, for example by a laboratory examination being carried out. Alternatively, a plurality of components having different LEDs would have to be used. With a plurality of LEDs, it is necessary for the latter to be successively switched on separately and thus for the wavelengths to be analyzed separately from one another. One spectral value is recorded per LED.

It is an object of the present invention, proceeding from the prior art described, to present a possibility of analyzing different constituents in milk with a low outlay.

This object is achieved by the apparatus, the arrangement and the method as claimed in the independent claims. Further advantageous configurations are specified in the dependent claims.

An apparatus for analyzing milk is presented according to the invention. The apparatus comprises:
a line portion for the milk,
a light source unit, which emits light into the line portion,
a detection unit for the spectrally resolved capture of light emerging from the line portion, and in particular
an evaluation unit configured to analyze the milk with regard to constituents on the basis of signals from the detection unit.

The apparatus described serves for analyzing milk obtained by milking, in particular cow's milk, which is analyzed indirectly or directly after a milking operation. In this case, milk should be understood to mean not only pure milk, but also contaminated milk. A mixture of pure milk and blood or chemicals such as cleaning agents or dipping agents constitutes contaminated milk and is also referred to as “milk” herein. An analysis should be understood to mean that the milk is examined with regard to its composition. In this case, the milk can be examined with regard to constituents such as fat, protein and lactose or with regard to contaminants such as antibiotics, dipping agents, cleaning agents or water. Moreover, it is possible to determine whether the milk contains solid particles, flakes, foam and bubbles. The analysis can consist in differentiating between the presence and absence of specific substances, without quantifying these substances. In this regard, it is possible to determine whether the milk has been contaminated. Alternatively or additionally, it is possible for example to establish what proportion of fat the milk has.

The absence of a specific constituent can be established in particular by virtue of this constituent not being present in the milk in an amount that is measurable by the apparatus. The milk can be examined for example in regard to whether the milk contains blood and/or urea. The milk contaminated with blood and/or urea can then be segregated, preferably before it is mixed with other milk. In addition, blood in milk can indicate an injury of the animal from which the milk originates. Such milk may need to be discarded on account of legal requirements. If blood is recognized, the corresponding animal can be subjected to an examination in order to verify the animal's state of health. Furthermore, the analysis can consist in quantifying constituents. In this regard, for example, the proportion of protein, fat and/or lactose can be ascertained.

The apparatus can contribute to process monitoring. By way of example, changes in the composition of the milk over time can be recognized by the apparatus. That is relevant particularly for the concentration of cleaning agents in the milk. The apparatus can recognize the lactation cycle or a gestation of an animal. In addition, the apparatus can be used for flow recognition. A distinction can thus be drawn for example between a milking operation and a cleaning operation. Preferably, the apparatus is configured to issue an action instruction in response to the determined results, for example in the form of an alarm or a warning.

The apparatus comprises a line portion for the milk. Line portion should be interpreted as part of a line through which the milk can flow. The line portion can be part of a pipe or tube, for example. The line portion has at least one inlet via which the milk can be introduced into the line portion, and at least one outlet via which the milk can pass out of the line portion. The at least one inlet and the at least one outlet are different from one another. The line portion does not have to be separately delimited from the rest of the line. In particular, it is not necessary for the line portion to extend over exactly one pipe component. The at least one inlet and the at least one outlet merely define the beginning and end, respectively, of the line portion and do not have to coincide for example with a joint between pipe components placed against one another. The line portion is defined physically only insofar as the apparatus is configured to analyze the milk within the line portion. That means that the line portion extends at least over the measurement region within which the milk can be analyzed. However, the measurement region need not encompass the entire line portion. In particular, it is possible for the measurement region not to extend over an entire line cross section.

The apparatus furthermore has a light source unit and a detection unit. By means of the light source unit, light can be guided into the line portion, for example via a window in a boundary of the line portion. The detection unit can detect light emerging from the measurement chamber, for example via a further window or the above-described window in the boundary of the line portion. The detection unit is preferably coordinated with the light source unit. Particularly preferably, the detection unit covers the entire spectral range emitted by the light source unit. The detection unit and the light source unit are preferably arranged in such a way that the light which emerges from the line portion and which originates from the light source unit can be detected by the detection unit. The detection unit has one or more detectors. The light source unit and the detection unit serve to spectroscopically analyze the milk. That is possible by virtue of the fact that the detection unit is configured for the spectrally resolved capture of the light. That means that the detection unit can capture the light intensity depending on the wavelength. The detection unit can thus record a multiplicity of individual spectral values, in particular from the entire wavelength spectrum of the light which emerges from the line portion and which originates from the light source unit. For the spectrally resolved capture of the light, the detection unit preferably comprises a means for spectrally decomposing the light, for example an interferometer or a dispersive element such as a grating or a prism. In the case of the dispersive element, the latter is preferably mounted rotatably. Alternatively, it is possible to use a spatially resolving detector which can measure the spectrally decomposed light at a measurement time, for example a CCD chip. In that case, the measurement accuracy arises in particular from the resolution of the CCD chip.

Preferably, the detection unit has a detector and an interferometer. An interferometer is an apparatus which generates interference by splitting a light beam into two partial beams and by combining the two partial beams with a path difference. The interferometer and the detector are designed and arranged in such a way that light emerging from the line portion can be spectrally decomposed by the interferometer and then detected by the detector. As a result of the spectral decomposition of the light by the interferometer, the detection unit can capture the light in a spectrally resolved manner. The interferometer thus makes it possible, in the case of the light emerging from the line portion, to determine a multiplicity of individual spectral values within the preferably continuous wavelength spectrum of the light source unit. The interferometer can be a Michelson interferometer or a Fabry-Perot interferometer, for example. Both of these interferometers have a movable mirror. Preferably, the interferometer has an, in particular exactly one, flexible element, which generates different subspectra of the total spectrum of the light source unit.

The detection unit can be automatically adjusted and/or calibrated by feedback of reference values. The detection unit can be adapted in a customer- and/or herd-specific manner. Furthermore, changes in the light source unit over time can be compensated for.

The apparatus is configured to analyze the milk within the line portion by virtue of the fact that the light source unit and the detection unit are aligned with the line portion. The measurement region for analyzing the milk that is formed by the light source unit and the detection unit falls within the line portion as a result. In this respect, the line portion is distinguished vis-à-vis other line parts.

The apparatus preferably comprises a microelectromechanical system, MEMS for short. A MEMS is a component having a movable microscopic structure. The latter can be actuated by mechanical loading or by application of an electrical voltage. In this regard, the detection unit can be realized by virtue of the fact that an interferometer having a movable mirror as such a microscopic structure is realized. The light source unit can be arranged in a fixed orientation with respect to the MEMS, for example in the form of a common component or in a common housing. The light source unit and the detection unit are preferably arranged in a fixed position and orientation relative to one another. Such an apparatus is particularly small, robust and easily integrable and enables comparatively simple analyses outside a laboratory. Moreover, such an apparatus can be produced comparatively simply and inexpensively in large numbers.

The fact that the apparatus has a light source unit means that one or more light source units are provided. The light source unit preferably comprises one or more light sources. If a light source unit has a plurality of light sources, the latter are preferably designed to be identical to one another. Alternatively, a continuous spectrum covering a desired spectral range can be obtained by means of LEDs coordinated with one another as light sources. A plurality of light sources can be arranged such that the entire measurement region is illuminated uniformly. The light sources can be of identical type or different. Combining different light sources makes it possible to obtain a particularly wide wavelength spectrum. It is also conceivable for the light source unit to have a plurality of discontinuous light sources such as, for example, vapor lamps, in particular sodium vapor lamps or mercury vapor lamps. Moreover, it is possible for the light source unit to be designed to the effect that an external radiation source is coupled in, for example via an optical waveguide. In that case, the light source unit is formed only by the optical waveguide.

The milk in the line portion can be analyzed by the apparatus described. The milk can thus be analyzed while the milk is flowing through the line portion. It is not necessary to take samples and analyze them. Taking samples would firstly be more laborious than analysis in a line portion with through-flow. Secondly, the results of an analysis of samples are typically available only with a delay. If the milk had already been mixed with other milk after taking the sample, the entire milk might therefore need to be disposed of. By contrast, the analysis in the line portion enables, by way of example, contaminated milk to be segregated particularly rapidly and simply, in particular before said milk is mixed with other milk. Moreover, the apparatus described enables a complete analysis of the milk, not just the analysis of a sample. By way of example, the apparatus can be used to analyze the milk directly after the milking, before the milk is mixed with other milk in a milk tank.

Preferably, the light source unit emits light with a continuous wavelength spectrum into the line portion. A continuous wavelength spectrum should be understood to mean that there is a wavelength range containing every wavelength in the light emitted by the light source unit. The wavelength spectrum thus has a portion without a gap in any case. That does not exclude the wavelength spectrum having a plurality of continuous portions with a respective gap between them. It is preferred, however, for the wavelength spectrum to be free of gaps overall. The wavelength spectrum is preferably a broadband continuous wavelength spectrum. The term “broadband” should be understood relative to the detection range of the detection unit. The wavelength spectrum preferably has wavelengths separated from one another by at least 200 nm, in particular by at least 500 nm. In that case, the wavelength spectrum covers at least a wavelength range which has a width of 200 nm or 500 nm and which contains every wavelength in the light. Particularly preferably, the wavelength spectrum covers at least the wavelengths in the range of 1350 to 2500 nm. The wavelength spectrum is preferably in the near infrared range and/or in the mid-infrared range. In that case, the analysis of the milk is infrared spectroscopy. However, it is also conceivable for the wavelength spectrum to entirely or completely cover the range of visible light and/or to entirely or completely cover the UV range. Particularly preferably, the wavelength spectrum covers a wavelength range that can be used to excite chemical bonds in the milk to be analyzed. In this regard, the so-called spectral fingerprint of the milk can be determined. The wavelength spectrum of the light emitted by the light source unit is preferably continuous in one portion in any case. By way of example, the wavelength spectrum can be a Planck's spectrum, as is present in black body radiation.

By means of the light source unit with a continuous spectrum, the milk can be analyzed with regard to different constituents. That can be done in particular in the manner of dispersive spectroscopy. The apparatus is therefore not restricted to the analysis of a single constituent. In the configuration of the apparatus, therefore, it is not necessary to fix the specification to a specific constituent. In this respect, the apparatus described is particularly flexible.

The apparatus furthermore preferably has an evaluation unit. The evaluation unit is configured to analyze the milk with regard to constituents on the basis of signals from the detection unit. The evaluation unit can be arranged in a housing together with the light source unit and the detection unit.

As an alternative or in addition to the use of an evaluation unit as part of the apparatus, the milk can also be analyzed outside the apparatus, for example by means of a central server and/or by means of a cloud application. The apparatus preferably has an interface via which the signals from the detection unit can be output, in particular to an evaluation unit configured to identify constituents of the milk on the basis of signals from the detection unit. The apparatus and the evaluation unit can be connected to one another via a cable, via a wireless connection and/or via an Internet connection.

For analyzing the milk, in particular by means of the evaluation unit, the signals emitted by the detection unit can be evaluated in the manner of dispersive spectroscopy. This is preferably done using a complex evaluation algorithm which can calculate the presence and optionally also the concentration of constituents of the milk. The evaluation algorithm uses the measured spectral information as input parameters and calculates therefrom the desired characteristic variables or values to be determined.

The evaluation algorithm can be obtained by a separate system with the aid of reference data and/or using a machine learning program. The signals emitted by the detection unit contain information concerning the light captured by the detection unit. Particularly in the case of a light source unit with a continuous spectrum, the apparatus can be switched over particularly easily to analyze other constituents. In particular, there is no need to change the hardware for this purpose. Instead it is sufficient to change the evaluation algorithm. Moreover, it is possible to adjust the measurement accuracy by adapting the software of the evaluation unit, for example in interplay with a measurement duration. The analysis can thus be changed by means of changing the software, for example by means of a software update. By means of a software update, it is possible in particular to extend the functional scope of the evaluation, for example by means of enabling a previously disabled function or a functional extension (actual addition of the function). The functioning of the evaluation can thus be changed without a construction change.

The evaluation is preferably performed in the manner of Fourier transform spectroscopy, in particular in the manner of Fourier transform infrared spectroscopy (FTIR for short). The constituents present in the milk can be recognized from the spectrum obtained in the process.

By way of example, it is possible to ascertain whether the spectrum has a peak at a characteristic wavelength of a specific constituent. Moreover, the constituents can be quantified. The height of a peak can be determined for this purpose.

The milk can be analyzed on the basis of the light reflected and/or absorbed by the milk. In order to use the reflected light, the light source unit and at least one detector of the detection unit are arranged on the same side of the line portion. The light from the light source unit can thus be guided into the line portion, be reflected by the milk in the line portion, and pass out of the line portion into the at least one detector of the detection unit. The light source unit and the detection unit can be arranged next to one another, for example within a common housing. This configuration is preferred owing to the possibility of this compact design. In this configuration, in particular, it is preferred for the apparatus to comprise a MEMS.

In order to use the absorbed light, the light source unit and at least one detector of the detection unit are arranged on mutually opposite sides of the line portion. In that case, the line portion is arranged between the light source unit and the at least one detector of the detection unit. The light from the light source unit can thus be guided into the line portion and, insofar as it is not absorbed by the milk in the line portion, it can pass out of the line portion into the detector.

If the detection unit has a plurality of detectors, these are preferably all arranged on the same side of the line portion. Thus, either the reflected light can be captured by all the detectors or the absence of the absorbed light can be captured by all the detectors. However, it is also conceivable for the detection unit both on the side of the light source unit and on the opposite side in each case to have one or more detectors. Both the reflected light and the absorbed light can be taken into account in that case.

The spectrum emitted by the light source unit can change over time. Therefore, it is preferred for the spectrum emitted by the light source unit to be measured as reference at regular intervals. Particularly preferably, a respective reference spectrum is recorded directly before each measurement for analysis purposes. If the analysis is performed on the basis of the reflected light, the spectrum measured for analysis purposes can be compared with a reference spectrum that was measured by an ideal reflector. If the analysis is performed on the basis of the absorbed light, the spectrum measured for analysis purposes can be compared with a reference spectrum that was measured with the line portion empty.

In one preferred embodiment, the apparatus furthermore has a main line, wherein the line portion branches off from the main line and leads into the main line.

The line portion runs parallel to the main line. As a result, this embodiment admittedly does not allow analysis of the entire milk. Nevertheless, the apparatus enables a more comprehensive analysis of the milk than the examination of individual visual samples. Finally, part of the milk flow can be examined thoroughly by the apparatus described.

The term “main line”, in delimitation from the term “branch section”, relates merely to the circumstance that the analysis of the milk takes place in the branched-off line portion and thus in a separate line part. It is not necessary for the main line to have a larger flow cross section than the branched-off line portion.

Upstream of the measurement region, the line portion preferably has a filter screen. The latter can be arranged for example at a branching-off point at which the branch section branches off from the main line. The filter screen enables solid particles of a corresponding minimum size to be kept away from the line portion. Blockage of the line portion can be prevented as a result.

In a further preferred embodiment of the apparatus, the branch section leads into the main line via a first opening and a second opening, wherein the first opening and the second opening are arranged at a distance from one another in a height direction.

The height direction relates to the orientation of the apparatus during use as intended. By virtue of the openings being arranged at a distance from one another in the height direction, the opening arranged further down can serve with priority for liquid components in the line portion, while gaseous components can pass with priority through the upper one of the openings back into the main line. This separation allows the liquid components to flow in the lower region of the line portion in a particularly undisturbed manner. The milk can therefore be analyzed with particularly high measurement accuracy. That is the case in particular in the preferred configuration in which the light source unit and the detection unit are arranged at the level of the lower half of the line portion. Particularly preferably, the light source unit and the detection unit are arranged below the line portion. This relates in each case to the arrangement of the light source unit and the detection unit in the height direction.

In a further preferred embodiment, an inlet and/or an outlet of the line portion are/is dimensioned in such a way that the flow of the milk through the line portion is delayed.

In this embodiment, the flow is at a lower level within the line portion than upstream and/or downstream thereof. A delay of the flow has an advantageous effect on the measurement and, in conjunction therewith, on the precision. Continuous exchange of liquid nevertheless takes place.

In a further preferred embodiment, the line portion is able to be shut off, in particular by a shut-off element within the line portion.

In this embodiment, the flow of milk can be completely stopped in order to carry out the analysis. The analysis takes place discontinuously. A particularly high measurement accuracy can be achieved as a result.

In a further preferred embodiment of the apparatus, the light source unit comprises a thermionic emission source.

Thermionic emission sources emit a continuous wavelength spectrum. Moreover, they are comparatively inexpensive. The thermionic emission source is preferably a halogen lamp. Such a lamp has a high and directional intensity.

An arrangement is presented as a further aspect of the invention. The arrangement comprises:
a milking device,
an apparatus for analyzing milk, comprising
   a line portion for the milk,
   a light source unit, which emits light into the line portion,
   a detection unit for the spectrally resolved capture of light emerging from the line portion,
   and in particular
   an evaluation unit configured to analyze the milk with regard to constituents on the basis of signals from the detection unit.

The milking device is connected to the line portion of the apparatus.

The described advantages and features of the apparatus are applicable and transferable to the arrangement, and vice versa. The apparatus contained in the arrangement is preferably designed like the apparatus described above.

The arrangement preferably serves for milking cows. The milking device is preferably designed as a milking cluster. Preferably, the arrangement has a milk tank, which is connected to the milking device via the line portion. The arrangement preferably comprises a plurality of milking devices connected to the milk tank. Before the milk passes from one of the milking devices into the milk tank, some of the milk is analyzed in any case. The milk can optionally be segregated before it is mixed with other milk in the milk tank. For the analysis, the arrangement comprises the above-described apparatus. The line portion of the apparatus can in this case be arranged at any arbitrary position between animal and milk tank.

Preferably, the apparatus has a milk tank which is connected to the milking device via the line portion. That means that a connection between the milk tank and the milking device comprises the line portion. However, the line portion need not extend from the milk tank as far as the milking device. It is even preferred for further elements, such as a milk sluice, for example, to be arranged between the milking device and the milk tank. Moreover, it is not necessary for the milk tank and the milking device to be connected to one another exclusively via the line portion. It is even preferred for a main line to be connected to the milk tank and the milking device, and for the line portion to be arranged in a branch section which branches off from the main line and which leads into the main line again.

By way of example, the line portion can be arranged such that the milk from a specific teat can be analyzed. Preferably, in that case, a respective apparatus is provided for each teat, such that an animal's milk can be analyzed teat-specifically. This analysis can also be referred to as quarter-specific in the case of cows. Alternatively or additionally, it is possible to arrange an apparatus with a line portion downstream of a milk collecting piece, such that the milk can be analyzed animal-specifically. Alternatively or additionally, it is possible to arrange an apparatus with a line portion between milking stall and milk tank. Alternatively or additionally, it is possible to arrange an apparatus with a line portion in the region of a junction combining the milk from a plurality of milking stalls. Alternatively or additionally, it is possible to arrange an apparatus with a line portion directly at the milk tank. It is also conceivable to arrange an apparatus with a line portion between an automatic cleaning system and a milking stall. An apparatus with a line portion can also be arranged in lines for milk that is not marketable.

The line portion is preferably able to be shut off. The fact that the milk tank is connected to the milking device via the line portion should be assessed independently of the position of a corresponding shut-off valve. In other words, the closing of a shut-off valve cannot deprive a line portion of the property thereof that the milk tank is connected to the milking device via said line portion.

A method for analyzing milk in a line portion is presented as a further aspect of the invention. The method comprises:
a) guiding light into the line portion,
b) spectrally resolved detection of light emerging from the line portion,
c) analyzing the milk with regard to constituents on the basis of the light detected in accordance with step b).

The described advantages and features of the apparatus and of the arrangement are applicable and transferable to the method, and vice versa. The method is preferably carried out by means of the apparatus described, and in particular by means of the arrangement described. The apparatus and the arrangement are preferably suitable for carrying out the method described.

It is sufficient to carry out each of steps a) to c) once. A snapshot can be obtained as a result. It is preferred, however, for each of steps a) to c) to be carried out a number of times. A series of spectra can thus be recorded and evaluated in each case. In this regard, the milk can be analyzed in particular at constant time intervals. As a result, it is possible to capture a temporal development during a milking operation.

In one preferred embodiment of the method, an evaluation algorithm is created by machine learning before step c), wherein the milk is analyzed using the evaluation algorithm in step c).

In order to create the evaluation algorithm, signals from the detection unit together with corresponding reference values are fed to the machine learning program. The reference values can be obtained by the milk analyzed as described being additionally analyzed by a laboratory examination, for example. Patterns between features of the signals from the detection unit and the reference values are recognized in this case.

The machine learning program can be parts of a separate device. In particular, the machine learning program can be installed on a computer that is not part of the apparatus described here. The machine learning program can be installed on a development tool, for example.

The evaluation algorithm can be created by the separate device and subsequently—if the apparatus has an evaluation unit—be communicated to the evaluation unit. Alternatively, the evaluation algorithm created by the separate device can be communicated by the separate device to a server used for analyzing the milk. This does not necessitate permanent contact between the separate device and the evaluation unit or the server. The signals from the detection unit can be communicated to the separate device in various ways, for example via the Internet or via a cable connection. If the apparatus has an evaluation unit, the evaluation algorithm created can be communicated to the evaluation unit in an identical way. The separate device can be spatially separated from the evaluation unit or can be arranged together with the latter in a common housing.

The evaluation algorithm can be created or changed by means of machine learning by the evaluation unit or a server itself that is used to analyze the milk, for example by the use of artificial intelligence.

It is sufficient for the evaluation algorithm to be created once. In this regard, by way of example, in a learning phase, it is possible to process a plurality of signals from the detection unit with a respectively corresponding reference value. It is preferred for the evaluation algorithm to be revised in particular at regular time intervals. For this purpose, in a new learning phase, a new evaluation algorithm can be created or the previous evaluation algorithm can be updated. The arrangement described preferably comprises a device for creating an evaluation algorithm from signals from the detection unit and corresponding reference values. The device preferably has a machine learning program. In that case, the evaluation unit is configured to analyze the milk on the basis of signals from the detection unit by applying the evaluation algorithm. A server that is simultaneously used for the analysis of the milk is also conceivable as the device.

Furthermore, the method preferably comprises:

d) outputting a signal if a constituent recognized in step c) exceeds a corresponding limit value.

Step d) makes it possible for example to give a farmer an action instruction regarding the intended reaction to the presence of a specific constituent. By way of example, the arrangement can have a display device used to display the action instruction in response to the signal output in step d). The signal output in step d) can alternatively or additionally be implemented in an automated manner. If blood, for example, is recognized in the milk, the milk contaminated in this way can automatically be segregated by a corresponding valve being switched in response to the signal output in step d). Additionally, the action instruction to examine the affected cow for an injury can be displayed to the farmer.

In one preferred embodiment of the method, the milk proceeding from a milking device is guided through the line portion.

In this embodiment, the milk can be analyzed particularly comprehensively and rapidly. This is possible in particular owing to the continuous wavelength spectrum of the light source unit.

In a further preferred embodiment of the method, step c) involves ascertaining whether at least one predefined constituent in the milk exceeds a respective limit value.

In this embodiment, the presence or absence of one or more specific constituents is examined. That is expedient for blood and urea as constituents, for example. In this case, a distinction is drawn between presence and absence on the basis of the predetermined limit value.

In a further preferred embodiment of the method, an evaluation algorithm used in step c) is changed.

In this embodiment, the described analysis of the milk is performed firstly by a first evaluation algorithm and then by a second evaluation algorithm. The evaluation algorithm can be changed from the first evaluation algorithm to the second evaluation algorithm by means of a software update, for example. The first evaluation algorithm and the second evaluation algorithm differ from one another, for example with regard to the detectable constituents of the milk and/or with regard to the achievable measurement accuracy.

In a further preferred embodiment of the method, the following steps are carried out cyclically:
A) collecting milk in the line portion,
B) analyzing the milk situated in the line portion in accordance with steps a) to c),
C) discharging the milk analyzed in step B) from the line portion.

This embodiment involves discontinuously analyzing the milk in the line portion. For this purpose, the milk is introduced into the line portion, and the line portion is shut off in such a way that the milk collects in the line portion (step A)). That can be done for example by the closing of a shut-off valve downstream of the line portion. As soon as a desired amount of the milk has collected in the line portion, this milk can be analyzed (step B)). This can be done particularly reliably because the milk is at rest during the measurement. Step B) is preferably begun only after step A) has finished. The measurement time is preferably between 0.5 and 5 seconds, in particular between 1 and 2 seconds. The line portion is preferably shut off during the measurement time. The milk is correspondingly at rest over the measurement time. After the analysis, the milk can be passed out of the line portion (step C)). For this purpose, for example, the shutting-off can be opened again. Step C) is preferably begun only when step B) has finished. Afterward, steps A) to C) are repeated. In this case step C) of a cycle can coincide with step A) of the subsequent cycle. In this regard, the analyzed milk can be passed from the line portion in a procedure in which the milk to be analyzed in the subsequent cycle is introduced into the line portion.

If the evaluation algorithm used in step c) is changed, this is preferably done between two cycles in this embodiment.

Steps A) to C) can be carried out successively as described. In this respect, the milk can be analyzed discontinuously. In an alternative embodiment, the milk is analyzed continuously. For this purpose, the milk can be continuously collected and discharged. This ultimately corresponds to restricting the flow of the milk. In this regard, an inlet and/or an outlet of the line portion can be dimensioned in such a way as to delay the flow of the milk through the line portion. A particularly high measurement accuracy can be achieved as a result.

The invention is explained in greater detail below with reference to the figures. The figures show particularly preferred exemplary embodiments, to which the invention is not limited, however. The figures and the size ratios illustrated therein are merely schematic, in which:

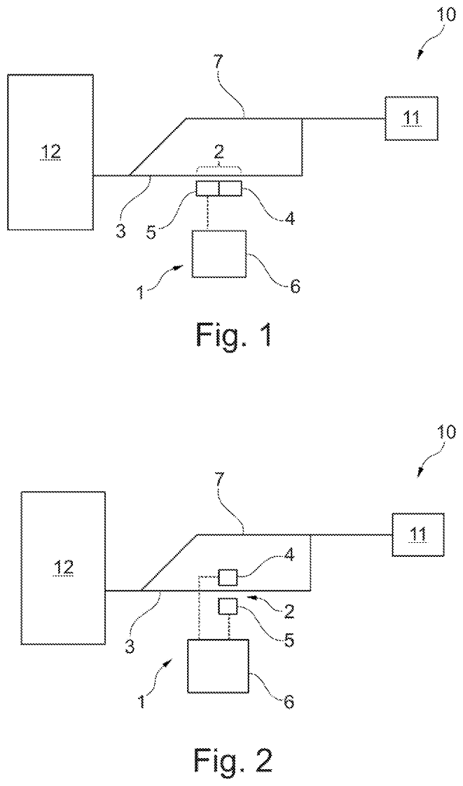

FIG. 1: shows a first embodiment of an arrangement according to the invention,

FIG. 2: shows a second embodiment of an arrangement according to the invention,

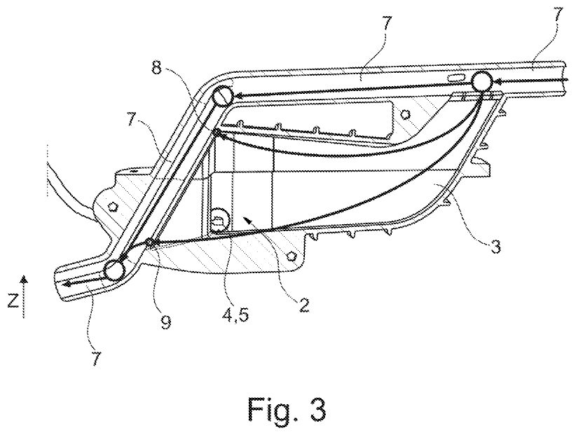

FIG. 3: shows a first embodiment of an apparatus according to the invention for analyzing milk,

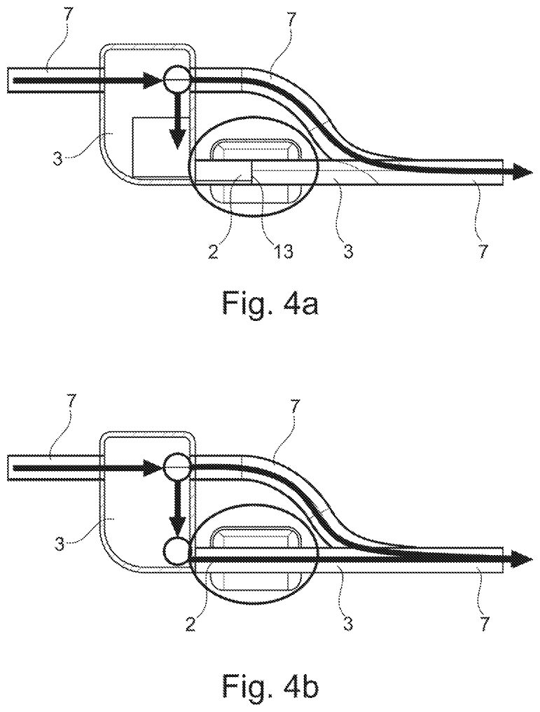

FIG. 4a, 4b: show a second embodiment of an apparatus according to the invention for analyzing milk,

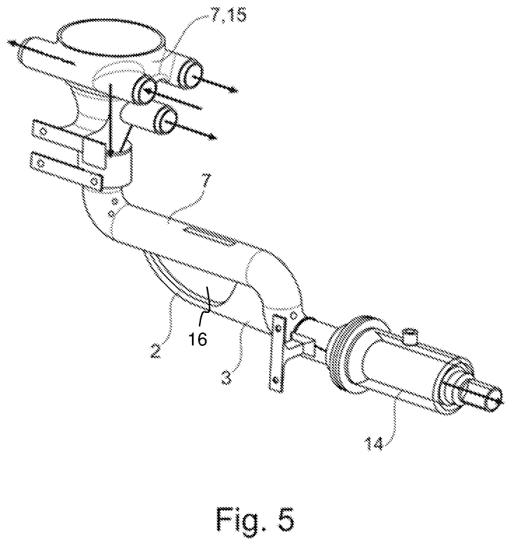

FIG. 5: shows a third embodiment of an apparatus according to the invention for analyzing milk,

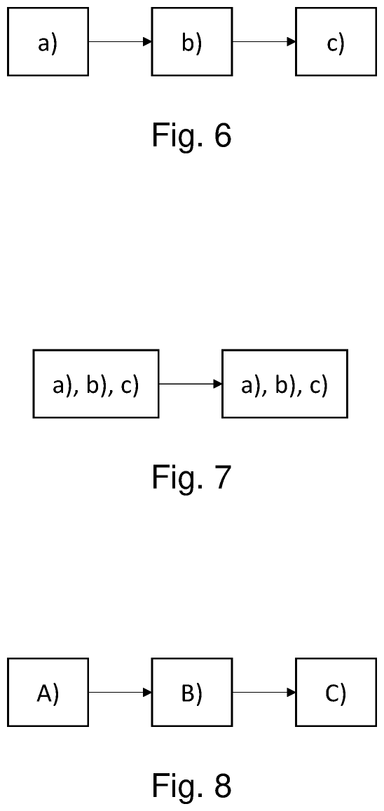

FIG. 6: shows a first flow diagram of a method according to the invention for analyzing milk,

FIG. 7: shows a second flow diagram of a method according to the invention for analyzing milk,

FIG. 8: shows a third flow diagram of a method according to the invention for analyzing milk.

FIG. 1 shows an arrangement 10 having a milking device 11 and a milk tank 12. The milking device 11 is connected to the milk tank 12 by a main line 7. A branch section 3 is provided parallel to the main line 7, said branch section branching off from the main line 7 and leading into the main line 7 again. The milk can be analyzed in a line portion 2 of the branch section 3. The milking device 11 is connected to the milk tank 12 via the branch section 3 and in this respect via the line portion 2. For the purpose of analyzing the milk the arrangement 10 has a halogen lamp as a light source unit 4, which emits light with a continuous wavelength spectrum into the line portion 2. Furthermore, the arrangement 10 has a detection unit 5 for the spectrally resolved capture of light emerging from the line portion 2. The detection unit 5 is connected to an evaluation unit 6 configured to analyze the milk with regard to constituents on the basis of signals from the detection unit 5. The milk can be analyzed when it is passed through the line portion 2 proceeding from the milking device 11. The analysis can involve ascertaining whether at least one predefined constituent in the milk exceeds a respective limit value. The line portion 2, the light source unit 4, the detection unit 5 and the evaluation unit 6 form an apparatus 1 for analyzing milk.

FIG. 2 shows an arrangement 10 similar to that from FIG. 1. It is just that here the light source unit 4 and the detection unit 5 are arranged on mutually opposite sides of the line portion 2. Consequently, the absorption of light can be measured in accordance with FIG. 2, while the reflection can be measured in accordance with FIG. 1.

FIG. 3 shows part of a first embodiment of an arrangement 10 according to FIG. 1 or 2. On account of the merely schematic illustration in FIGS. 1 and 2, various embodiments of milking stalls 10 can be designed according to FIGS. 1 and 2. The main line 7 with a branch section 3 is shown. The light source unit 4 and the detection unit 5 are arranged in such a way that it is possible to analyze the milk in part of the branch section 3. The branch section 3 constitutes a line portion 2. The branch section 3 leads into the main line 7 via a first opening 8 and a second opening 9. The first opening 8 is arranged higher than the second opening 9 in a height direction z. A gaseous component can pass into the main line 7 from the branch section 3 via the first opening 8, while a liquid component can pass into the main line 7 from the branch section 3 via the second opening 9.

FIGS. 4a and 4b show part of a second embodiment of an arrangement 10 according to FIG. 1 or 2. The main line 7 is shown, with through-flow from left to right in this illustration. The line portion 2 in the branch section 3 is able to be shut off in this configuration. A shut-off element 13 is provided for this purpose. The latter is closed in FIG. 4a. As a result, the milk can be collected in the line portion 2 and analyzed therein. After the analysis has finished, the shut-off element 13 can be opened and the analyzed milk can thus be passed from the line portion 2. At the same time, new milk can flow into the line portion 2. Renewed closing of the shut-off element 13 enables this new milk to be collected in the line portion 2. These steps can be repeated cyclically.

FIG. 5 shows part of a third embodiment of an arrangement 10 according to FIG. 1 or 2. The main line 7 is shown, which here comprises a cyclone geometry 15 and is attached to a valve 14 for a teat cup liner. The valve 14 is part of the milking device 11. The cyclone geometry 15 serves for phase separation, for example of air, foam and milk, and to delay the flow. The cyclone geometry 15 has at least one inlet and at least one outlet. A measurement region 3 in a line portion 2 is arranged below the main line 7. The measurement region 3 is optically accessible from the outside via a transparent window 16.

FIG. 6 illustrates a method for analyzing milk in a line portion 2, which method can be carried out by means of the apparatuses 1 and arrangements 10 shown above. The method comprises:
a) guiding light into the line portion 2,
b) spectrally resolved detection of light emerging from the line portion 2,
c) analyzing the milk with regard to constituents on the basis of the light detected in accordance with step b).

FIG. 7 illustrates one embodiment of the method from FIG. 6, wherein the evaluation algorithm is changed. In this embodiment, the described analysis of the milk is performed firstly by a first evaluation algorithm and then by a second evaluation algorithm.

FIG. 8 illustrates a further embodiment of the method from FIG. 6, wherein the following steps are carried out cyclically:
A) collecting milk in the line portion 2,
B) analyzing the milk situated in the line portion 2 in accordance with steps a) to c),
C) discharging the milk analyzed in step B) from the line portion 2.

### LIST OF REFERENCE SIGNS

1 Apparatus
2 Line portion
3 Measurement region
4 Light source unit
5 Detection unit
6 Evaluation unit
7 Main line
8 First opening
9 Second opening
10 Arrangement
11 Milking device
12 Milk tank
13 Shut-off element
14 Valve
15 Cyclone geometry
z Height direction

1. An apparatus for analyzing milk, comprising:
a line portion for the milk;
a light source unit, which emits light into the line portion;
a detection unit for the spectrally resolved capture of light emerging from the line portion;
and
an evaluation unit configured to analyze the milk with regard to constituents on the basis of signals from the detection unit.

2. The apparatus as claimed in claim 1, furthermore having a main line, wherein the line portion branches off from the main line and leads into the main line.

3. The apparatus as claimed in claim 2, wherein the line portion leads into the main line via a first opening and a second opening, and wherein the first opening and the second opening are arranged at a distance from one another in a height direction.

4. The apparatus as claimed in claim 1, wherein the light source unit comprises a thermionic emission source.

5. An arrangement, comprising:
a milking device;
an apparatus for analyzing milk, the apparatus including:
a line portion for the milk;
a light source unit, which emits light into the line portion;
a detection unit for the spectrally resolved capture of light emerging from the line portion; and
an evaluation unit configured to analyze the milk with regard to constituents on the basis of signals from the detection unit,
wherein the milking device is connected to the line portion of the apparatus.

6. The arrangement as claimed in claim 5, wherein the apparatus furthermore has a main line, wherein the line portion branches off from the main line and leads into the main line.

7. A method for analyzing milk in a line portion, comprising:
a) guiding light into the line portion,
b) spectrally resolved detection of light emerging from the line portion,
c) analyzing the milk with regard to constituents on the basis of the light detected in accordance with step b).

8. The method as claimed in claim 7, wherein an evaluation algorithm is created by machine learning before step c), and wherein the milk is analyzed using the evaluation algorithm in step c).

9. The method as claimed in claim 7, wherein the milk proceeding from a milking device is guided through the line portion.

10. The method as claimed in claim 7, wherein the following steps are carried out cyclically:
A) collecting milk in the line portion;
B) analyzing the milk situated in the line portion in accordance with steps a) to c); and
C) discharging the milk analyzed in step B) from the line portion.
